(19) United States
(12) Patent Application Publication
(10) Pub. No.: US 2006/0058701 A1
(43) Pub. Date: Mar. 16, 2006

(54) Systems and methods for providing sensory input

(75) Inventors: Mary Louise Bolles, Longmont, CO (US); Wayne Douglas Joseph Picotte, Longmont, CO (US)

Correspondence Address: FAEGRE & BENSON LLP;PATENT DOCKETING, 2200 WELLS FARGO CENTER, 90 SOUTH 7TH STREET, MINNEAPOLIS, MN, 55402-3901, US

(73) Assignee: Sensory Learning Center International, Inc., Boulder, CO (US)

(21) Appl. No.: 10/940,260

(22) Filed: Sep. 13, 2004

(51) Int. Cl. A61B 13/00; A61B 5/00

(52) U.S. Cl. 600/558; 600/559

(57) ABSTRACT

Various systems and methods for introducing multi-sensory stimuli to a participant and/or evaluating a participant's reaction to a delivered stimuli. As just one example, an embodiment of the present invention includes a trochoidal movement table, a light emitting visual display, an audio device and a controller. The controller includes a processor and a computer readable medium, and the controller is coupled to the trochoidal movement table and the light emitting display. The controller is operable to receive a sensory program from a remote server and to store the sensory program to the computer readable medium. The sensory program includes instructions executable by the processor to cause the trochoidal movement table to move in one of at least two selectable directions, and to cause the light emitting visual display to emit a visual stimulus.

### BACKGROUND OF THE INVENTION

The present invention relates generally to systems and methods for exposing a participant to various sensory stimuli, and in particular to systems and methods for delivering one or more sensory programs.

In various situations it is desirable to deliver stimuli to a participant and to evaluate the participant's reaction to the stimuli. Some devices have been developed for this purpose. For example, a screening audiometer can be used to determine the listening level of a human being. Such systems are typically limited to evaluating only certain senses by a professional local to the participant being evaluated. Indeed, many devices useful in evaluating a reaction to stimuli are not capable of delivering multiple stimuli. In at least one case, a system has been developed that tests a participants reaction to multiple stimuli including sound, motion and visual stimuli. Effective operation of such a system, however, requires a relatively highly skilled professional at the same location as the system. Among other things, this requirement can limit access to evaluations using the system, can improperly allow use of the system, can limit the full range of known system uses, and can result in the improper function of the system.

Hence, for at least the aforementioned reasons, there exists a need in the art for advanced systems and methods for introducing multiple stimuli to a participant and/or for evaluating a reaction of the participant to the multiple stimuli.

### BRIEF SUMMARY OF THE INVENTION

Various embodiments of the present invention provide systems and methods for introducing sensory input to a participant, and in particular one or more embodiments of the present invention provide systems and methods for introducing sensory input in a controlled fashion to a plurality of human sensory receptors and for controlling such introduction remotely. In some cases, such embodiments include introducing motion, sound and visual senses in a carefully synchronized presentation capable of inducing a participant reaction without over stimulating the participant.

table to move at a defined rate; cause the audio device to emit an audible stimulus; and cause the light emitting visual display to emit a visual stimulus.

Yet other embodiments of the present invention provide methods for delivering a multi-sensory experience. The methods include providing a movement table, providing a light emitting visual display, providing an audio device, and providing a controller. The controller is electrically connected to the movement table, the audio device, and the light emitting visual display. The methods further comprise delivering a command to the controller via a communication network. The command at least in part controls the stimuli delivered by at least one of the movement table, the light emitting display, and the audio device. Further, in some cases, the command is operable to preclude use of at least one of the movement table, the light emitting visual display and the audio device. In other cases, the command is operable to cause a modulated musical sound to emanate from the audio device, while in yet other cases the command is operable to cause a series of uniform colors to display via the light emitting display and/or to control a rate at which the movement table moves.

Additional embodiments of the present invention provide multi-sensory introduction systems. The systems include a movement table that is operable to introduce a movement sense to a participant disposed on the table, an auditory input device that is operable to introduce an audio sense to the participant, and a visual input device that is operable to introduce a visual sense to the participant. The systems further include a controller that is communicably coupled of the movement table, the auditory device, and the visual input device. The controller is operable to receive a command or operational key associated with the at least one of the movement table, the auditory device, and the visual input device from a remote server. In some cases, the movement table is a trochoidal motion table that is controlled by the controller and the movement sense is produced in accordance with the table motion under the direction of the controller. In operation, such a trochoidal motion table ca be utilized to maintain the head of the participant fixed in relation to the body of the participant.

In some cases, the auditory input device is further operable to receive a song and to segregate the song into at least one song segment, and wherein the audio sense introduced to the participant includes the at least one song segment. In some cases, the song segment or segments are created by attenuating the sound level during portions of the song such that the sound output is segmented. This segmentation can include partially attenuating the output level of the sound such that it is still audible and/or attenuating portions of the song such that it becomes inaudible. Thus, a segment can be a portion of a song played at one output level and terminating when the output level is modified to play at a different output level. Based on the disclosure provided herein, one of ordinary skill in the art will recognize various song segments that can be created and used in accordance with embodiments of the present invention. In various cases, a song is received by the auditory input device and output such that the song is presented as a modulated musical sound which is then introduced to the participant. In some cases, the modulated musical sound is created by attenuating the output level of portions of the song relative to other portions of the song. In some instances, the song is received by the controller from the remote server.

In various cases, the visual input device is further operable to receive a light sequence command, and the visual sense includes one or more light outputs presented in accordance with the light sequence command. In one particular instance, the light sequence command is received from the remote server. Further, in some cases, the controller is further operable to prevent operation of at least one of the movement table, the auditory device, and the visual input device in accordance with a operational command. In some instances, an operational command preventing operation is received from the remote server.

Yet other embodiments of the present invention provide server based sensory introduction systems. Such systems include a server communicably coupled to a remote multi-sensory introduction system. The server includes a processor and a computer readable medium, and the computer readable medium includes instructions executable by the processor to: select a sensory program that includes at least two sensory commands, and communicate the sensory program to the remote multi-sensory introduction system. In some cases, the computer readable medium further includes instructions executable by the processor to: check a compliance characteristic of the remote multi-sensory introduction system, and communicate an operational command to the remote multi-sensory introduction system based at least in part on the compliance characteristic. Such a compliance characteristic can be, but is not limited to, a license fee status, an operational status, and/or the like. In other cases, the computer readable medium further includes instructions executable to select an additional sensory program and to communicate the additional sensory program to the remote multi-sensory introduction system. In various cases, the computer readable medium further includes instructions executable by the processor to: receive a participant characteristic, and to select a sensory program based at least in part on the received participant characteristic. In some instances, the received participant characteristic is evaluated after a sensory program is applied to the participant (i.e., a post-sensory characteristic), and in other instances the received participant characteristic is evaluated before a sensory program is applied to the participant (i.e., a pre-sensory characteristic).

This summary provides only a general outline of some embodiments of the present invention. Many other objects, features, advantages and other embodiments of the present invention will become more fully apparent from the following detailed description, the appended claims and the accompanying drawings.

### BRIEF DESCRIPTION OF THE DRAWINGS

A further understanding of the various embodiments of the present invention may be realized by reference to the figures which are described in remaining portions of the specification. In the figures, like reference numerals are used throughout several to refer to similar components. In some instances, a sub-label consisting of a lower case letter is associated with a reference numeral to denote one of multiple similar components. When reference is made to a reference numeral without specification to an existing sub-label, it is intended to refer to all such multiple similar components.

FIG. 1 illustrates a multi-sensory introduction system in accordance with some embodiments of the present invention;

FIG. 2 is a block diagram of a remotely accessible multi-sensory introduction system in accordance with some embodiments of the present invention;

FIG. 3 is a flow diagram illustrating a method for delivering sensory stimuli in accordance with various embodiments of the present invention;

FIG. 4 is a flow diagram illustrating a method for delivering sensory stimuli in relation to an evaluation according to some embodiments of the present invention; and

FIG. 5 is a flow diagram illustrating a method for remote use prevention in accordance with one or more embodiments of the present invention.

### DETAILED DESCRIPTION OF THE INVENTION

Various embodiments of the present invention provide systems and methods for introducing sensory input to a participant, and in particular one or more embodiments of the present invention provide systems and methods for introducing sensory input in a controlled fashion to a plurality of human sensory receptors and for controlling such introduction remotely. In some cases, such embodiments include introducing motion, sound and visual senses in a carefully synchronized presentation capable of inducing a participant reaction without over stimulating the participant.

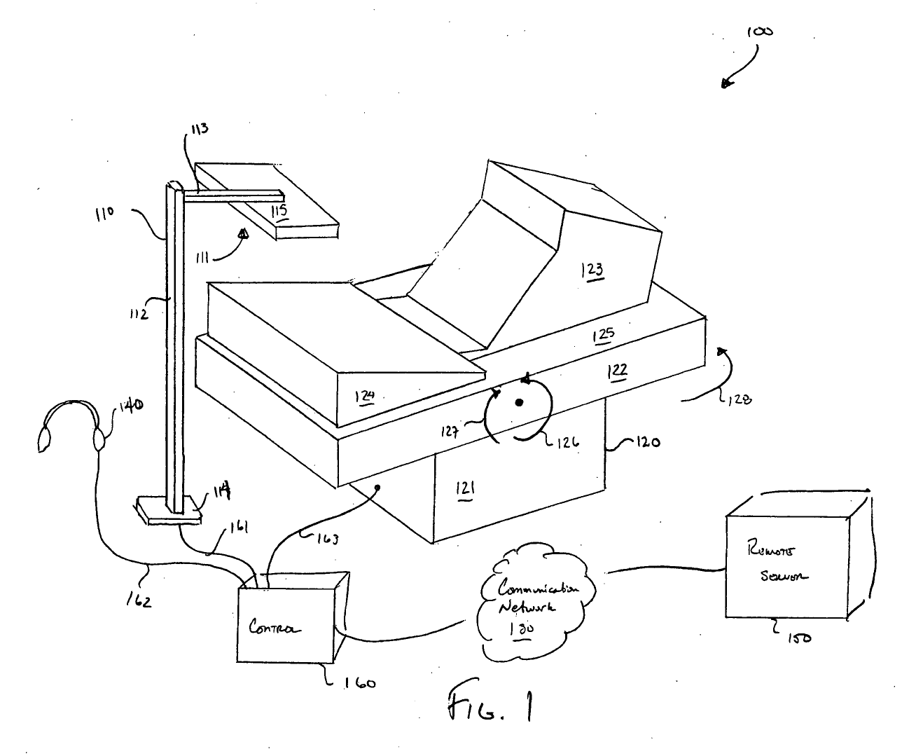

Turning to FIG. 1, a multi-sensory introduction system 100 in accordance with some embodiments of the present invention is depicted. System 100 includes a visual input device 110, a movement table 120, an auditory input device 140, a remote server 150 and a controller 160. Controller 160 is communicably coupled to remote server 150 via a communication network 130. Further, controller 160 is communicably coupled to auditory input device 140, movement table 120, and visual input device 110 via wires 162, 163, 161, respectively. As used herein, the term “communicably coupled” is used in its broadest sense to include any connection or method whereby information can be transmitted between devices. Thus, for example, devices can be communicably coupled via a wire, an optical coupling, an RF or other wireless coupling, and/or the like. As used herein, the term “electrically coupled” is a subset of communicably coupled and implies the use of a wire extending between coupled devices. Also, as used herein, the term “communication network” is used in its broadest sense to mean any network whereby information can be passed. Thus, for example, communication network 130 can be the Internet, a PSTN, a virtual private network, a peer to peer network, a WAN, a LAN, and/or any combination thereof or the like. While the exemplary system of FIG. 1 shows wired coupling between controller 160 and various devices and communication network 130, one of ordinary skill in the art will recognize that various media can be used to provide the various communicable couplings.

Movement table 120 can be any table capable of moving a participant disposed on the table through space. In some embodiments of the present invention, movement table 120 is a trochoidal movement table capable of providing a smooth movement pattern. In some instances, the movement pattern is a round or smooth movement pattern that avoids sudden changes in direction. In one particular instance, the movement pattern is a circular movement pattern in either a clockwise rotation as indicated by directional arrow 127, or a counter-clockwise rotation as indicated by directional arrow 126. The rate of movement can be adjusted such that a participant disposed on movement table 120 does not perceive that they are moving, but rather perceives that stationary objects around the participant are moving. In one particular case, the movement table is adjusted such that it moves along a circular path with a diameter of between four and eleven inches and at a rate of between seven and fourteen revolutions per minute. In one particular case, the table rotates along a circular path with a diameter of seven inches and at a rate of ten to eleven revolutions per minute and is capable of inducing vestibular stimulation in an anterior/posterior orientation (e.g., similar to doing somersaults one after another) and a lateral orientation (e.g., similar to rolling over and over like a log). In some cases, this motion can arouse the reticular activating system of a participant's brain. The table top may be rotated ninety degrees as indicated by an arrow 128 so that a participant is exposed to anterior/posterior movement in one session and lateral the next.

As illustrated, movement table 120 can have a moving portion 122 including a table top 125 that is connected to a fixed base 121. A motor is included that causes moving portion 122 to move in relation to fixed base 121. In some cases, moving portion 122 can be rotated ninety degrees (as indicated by arrow 128) relative to fixed base 121 to create either an anterior/posterior or lateral movement pattern. In some cases, foam pads 123, 124 are disposed on table top 125 to aid in maintaining the position and/or comfort of a participant disposed on movement table 120. In another particular embodiment of the present invention, the location of a persons head in relation to their body is maintained while the participant is moved through space. Maintaining the head fixed in relation to the body, providing a particular head tilt relative to the body, and/or maintaining the tilt of the combined body and head may aid in assuring that a participant positioned on movement table 120 perceives any movement to be associated with fixed objects around the participant (e.g., visual input device 110).

Visual input device 110 can be any device capable of displaying a controlled light pattern and creating a visual sense for a participant disposed on movement table 120. In one particular embodiment, visual input device 110 includes a circular opening or aperture through which light is passed. In such an embodiment, a series of colored light can be passed through the circular opening. This series of colored lights can be, for example, ruby, red, yellow-green, blue-green, violet and magenta with each color displaying in series for a defined period of time. In some cases, this series of colors can be presented automatically as dictated by a sensory program executed by, for example, controller 160. Examples of such control can be provided via a sensory program which is discussed below in more detail. In some cases, visual input device 110 is stationary relative to moving portion 122 of movement table 120 such that a participant disposed on movement table 120 can be moved through space relative to the stationary visual input device 110.

In one particular embodiment of the present invention, visual input device 110 includes an aperture of between one and four inches in diameter through which light is displayed to a participant. In one particular case, the aperture has a diameter approximately the same size as a golf ball. In some cases, the aperture is disposed between fifteen and twenty-two inches above a participant's eyes when the participant is disposed on movement table 110. In one particular instance, the aperture is disposed between seventeen and twenty inches above a participant's eyes when the participant is disposed on movement table 110. Providing the visual stimulation to a participant may, for example, trigger sympathetic and parasympathetic systems and changes in the firing pattern of the hypothalamus of the participant when visual light on the red end of the spectrum is presented, and a sedating effect on the blue end of the spectrum. Further, providing the visual stimulation may exercise the extrinsic eye muscles and strengthen and improve the ability of the eyes to track and team together. Further, providing the visual and movement stimuli together may aid the participant in adapting to the combination of stimuli and strengthen the vestibular ocular reflex. Also, the red end of the spectrum can trigger the extrinsic eye muscles to shape the eyeball as if looking in the near point and the blue end of the spectrum triggers the muscles as if looking in a distance. Such changes may excercise a participant's accommodation skills.

As illustrated, visual input device 110 includes a base 114 supporting a stand 112 and a connector 113. Connector 113 attaches to a light head 115 with an opening 111 through which light is shown. It should be noted that visual input device 110 can be formed in various shapes and sizes. For example, in one embodiment, stand 112 is attached to a non-moving portion of movement table 120.

Auditory input device 140 can be any device capable of outputting an audible signal to a participant. In some embodiments of the present invention, auditory input device 140 is capable of receiving a song which can be any series of audible notes, and modulating the song. In some cases, the modulation is done by a software program executed by a microprocessor. The software program reduces the output level or volume that is presented to the participant at varying intervals during the song. Thus, for example, three seconds of the song may be played at a first volume level, a subsequent second of the song can be played at a reduced volume level, and a subsequent two seconds of the song can be played at the first volume level. In some cases, the reduced volume level is not audible by the participant, while in other cases it is audible. This modulation creates song segments. Further, the modulation can be applied with a number of different volume levels, or only with two volume levels. Yet further, the modulation can be applied at random time orders and/or random volume levels, or can be presented in a non-random predictable sequence or some combination of random and non-random. In one particular case, elements of a song between 20 and 1000 Hz are modulated in one way and elements of the song between 1000 and 20000 Hz are modulated in another way. The separately modulated song portions can then be recombined to create a single modulated musical song. This modulated musical song can then be presented to a participant to create an audio sense.

Controller 160 can be any microprocessor based device capable of communicating with remote server 150 and issuing commands to one or more of movement table 120, visual input device 110, and auditory input device 140. In one particular embodiment, controller 160 is a personal computer (PC) capable of executing one or more software programs that result in control signals. For example, one software program can be a sensory program that directs the introduction of various sensory input to a participant via one or more of movement table 120, visual input device 110, and auditory input device 140. Alternatively, or in addition, software can be included to control the operational status of one or more of movement table 120, visual input device 110, and auditory input device 140. Thus, for example, it may be determined by remote server 150 that software license fees have not been paid and that operation of the system no longer exists. In such a case, remote server 150 may communicate a command that is executed by controller 160 to stop operation of the effected system portions.

Remote server 150 can be any microprocessor based system capable of communicating with controller 160 via communication network 130. Thus, as just some examples, remote server 150 can be a PC, a network server, or the like. Based on the disclosure provided herein, one of ordinary skill in the art will recognize that remote server 150 can be a single unified device such as a PC, or can be a number of microprocessor based devices each performing one or more functions attributed to remote server 150. Similarly, remote server 150 can be a single unified device, or a distributed group of devices with each performing different functions.

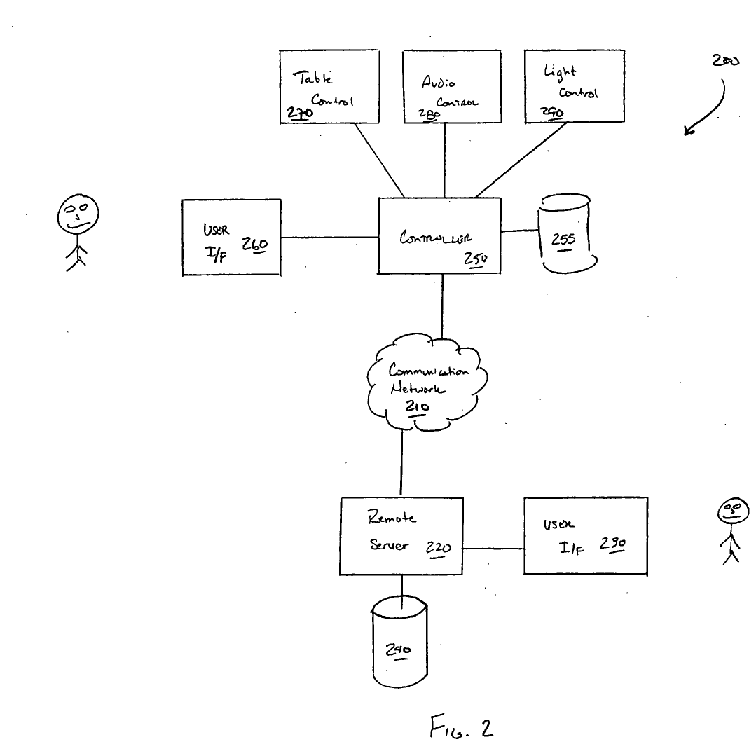

Turning to FIG. 2, a block diagram of a remotely accessible multi-sensory introduction system 200 in accordance with some embodiments of the present invention is illustrated. System 200 includes a control system 250 communicably coupled to a table control system 270, an audio control system 280 and a light control system 290. Table control system 270, audio control system 280 and light control system 290 can be any system or device capable of providing control commands to movement table 120, auditory input device 140 and visual input device 110, respectively. In some cases, the control systems 270, 280, 290 are implemented with both hardware and software, while in other cases they are implemented in either hardware or software. Further, in some cases, the control systems 270, 280, 290 are incorporated into the device or system that they control. In other cases, the control systems 270, 280, 290 are incorporated at least in part with control system 250. In one such case, control system 250 includes software corresponding to each of control systems 270, 280, 290 and hardware corresponding to each of control systems 270, 280, 290 is implemented in respective ones of movement table 120, auditory input device 140 and visual input device 110. Control system 250 can be distributed such that it is implemented as part of a number of devices, or implemented as part of a single device such as a PC.

Control system 250 is communicably coupled to a computer readable medium 255. Computer readable medium 255 can be any medium that is accessible to control system 250 and capable of storing instructions executable by the processor(s) of control system 250 and/or capable of maintaining data accessible to control system 250.

Control system 250 is communicably coupled to a remote server 220 via a communication network 210. As just some examples, communication network 210 can be the Internet, a PSTN, a virtual private network, a peer to peer network, a WAN, a LAN, and/or any combination thereof or the like. Remote server 220 can be any microprocessor based system capable of communicating with control system 250 via communication network 210. Thus, as just some examples, remote server 220 can be a PC, a network server, a combination of PCs and/or network servers, or the like.

Control system 250 may be accessible via a user interface 260. User interface 260 can be a graphical interface, a keyboard, a mouse, some combination of the aforementioned devices and/or other interface devices known in the art. User interface 260 can be capable of receiving information from a user and providing it to control system 250. Such information can be, for example, manual commands for operating one or more of movement table 120, auditory input device 140 and visual input device 110. Further, such information can be evaluation information about or characteristics of a participant exposed to various stimuli. Based on the disclosure provided herein, one of ordinary skill in the art will recognize a number of other information that can be received and portrayed via user interface 260.

Remote server 220 can be communicably coupled to a computer readable medium 240 and a user interface 230. Computer readable medium 240 can be any medium that is accessible to remote server 220 and capable of storing instructions executable by the processor(s) of remote server 220 and/or capable of maintaining data accessible to remote server 220. User interface 230 can be a graphical interface, a keyboard, a mouse, some combination of the aforementioned devices and/or other interface devices known in the art. User interface 230 can be capable of receiving information from a user and providing it to remote server 220. Such information can be, for example, commands indicating one or more sensory programs to be executed by one or more of control system 250, table control system 270, audio control system 280, and light control system 290. Further, user interface 230 can be tailored for displaying any evaluation information received about a participant exposed to various stimuli. Based on the disclosure provided herein, one of ordinary skill in the art will recognize a number of other information that can be received and portrayed via user interface 260.

Remote server 220 may be used for monitoring functionality of a remotely located sensory input system where software facilitating this operation is installed on remote server 220 and/or controller 250. Thus, for example, a remote server may make it possible to debug a sensory input system by a technician acting across a communication network. This can be an advantage where a number of sensory input systems are deployed, and it is desirable to limit the amount of maintenance costs expended in relation to the systems. Alternatively, or in addition, remote server 220 may be used to preclude operation of a sensory input system when conditions dictate such. Thus, as just one example, a remote kill switch can be installed whereby an operational command is issued from remote server 220 that precludes operation of the sensory input system. The operational command may be executed by one or more of controller 250, table control system 270, audio control system 280, and light control system 290. Also as an addition or an alternative to any or all of the aforementioned uses, remote server 220 can be used to accept characteristics of a participant, and allow a trained professional to evaluate the characteristics and select an appropriate sensory program for use with the participant. This allows for the trained professional to be located remote from the sensory input system to dictate or interact in the operation of the system. This can be particularly advantageous where selecting a sensory program for use in relation to the system involves considerable training. Based on the disclosure provided herein, one of ordinary skill in the art will recognize a number of other uses and/or advantages that may be had through use of a remote server in relation to a sensory input system.

In one particular application of a sensory input system in accordance with an embodiment of the present invention, the system is used to provide multiple sensory stimuli to a participant. In some cases, the participant can be a human that has a somewhat limited ability to combine multiple stimuli into a single experience. In some cases, such a human may suffer from what is commonly referred to as autism. Evaluating and/or treating autism is more fully described in the following two articles, the entirety of which are incorporated herein by reference for all purposes: “Cortical Activation and Synchronization During Sentence Comprehension in High-Functioning Autism: Evidence of Underconnectivity”, Just et. al., BRAIN a Journal of Neurology Vol. 127, No. 8; “Merging Sensory Signals in the Brain: The Development of Multisensory Integration in the Superior Colliculus”, Stein et al., Development of the New Cognitive Neurosciences, MIT Press, Cambridge, Mass., 2^{nd} ed. (2000).

The sensory input system provides a tool for the controlled introduction of multiple sensory stimuli in such a way that the participant is not overwhelmed. This allows the participant to, in a sense, practice incorporating multiple stimuli and in some cases gain a greater ability to cope with complex situations. This operates to provide an educational experience for the participant through the integrated and controlled use of multiple sensory stimuli including, but not limited to, visual colored light, modulated musical sound, and vestibular stimulation (e.g., motion). Said another way, such a sensory system can help a human organize their sensations. In some cases, the auditory input device provides modulated musical sound. The recurring attenuation in the modulated musical sound makes it difficult for the participant to become secure in the musical tune, thus continually bringing the participant present by helping the participant to shift attention from point to point. Further, in some cases, a higher volume is introduced to one of the participant's ears relative to the other. In some cases a higher volume is introduced to the right ear than to the left which may help to improve a participant's right ear dominance.

Further, in some cases, listening characteristics of a participant (i.e., one example of participant characteristics) are measured or otherwise obtained. This can include measuring the amplitude and/or frequency of an audible output that can be detected by a participant. This information can be assembled into a listening profile indicating a number of characteristics of the participant's listening abilities. In some cases, a screening audiometer can be used to develop the listening profile. It may be that the listening profile is more representative of a participant's ability to maintain attention and process auditory stimuli than the participant's sensitivity to auditory stimuli. Thus, in some cases, the listening profile may be an indication of the any uneveness between ears in a participant's hearing thresholds. Based on the disclosure provided herein, one of ordinary skill in the art will recognize a number of possible approaches for identifying a participant's listening characteristics and/or assembling a listening profile.

In various cases, the visual characteristics (i.e., one example of participant characteristics) of a participant are also measured or otherwise obtained. In so doing, a visual field chart can be completed upon exposing a participant to various colored (i.e., frequencies of visual light) one half degree targets presented at various angles relative to the participant. For evaluation, a participant is instructed to centralize their vision at a central point. At this time three wands or targets (red, blue, green) stimulating color cones of the participant's eye are slowly moved toward the central point from the periphery. When the participant indicates that they can see the wand, a color mark indicating the wand seen is made in each of eight pie-shaped segments on a chart. This colored chart is in essence a perception map of the back of the participant's eye (i.e., the fovea, the periphery and the area around the blind spot). These colored marks provide an indication of a neurotypical amount of photocurrent traveling along the optic nerve or whether a restrictive amount is traveling along the optic nerve. A constricted amount as indicated by a limited field of view may indicate a learning impediment associated with the visual system of the participant. A motion field can also be obtained using a white target in a fashion similar to that previously described. Likewise, a white target can be used to determine if any swelling around the optic nerve is evident as indicated by an abnormally large blind spot.

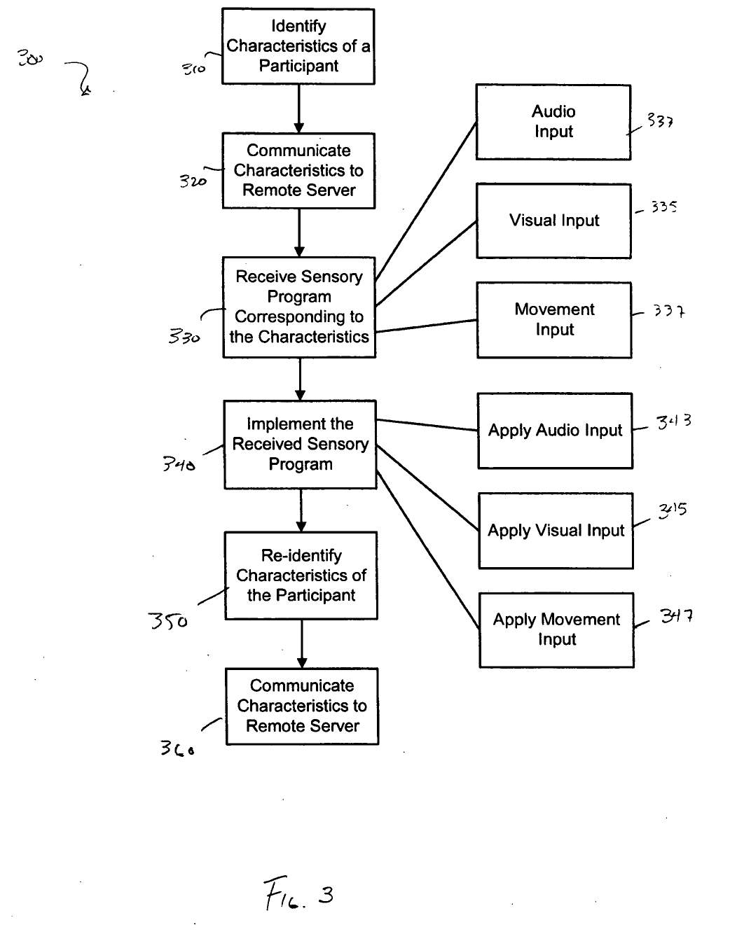

Turning to FIG. 3, a flow diagram 300 illustrates a method for delivering sensory stimuli. Following flow diagram 300, a participant characteristic is identified (block 310). Such characteristics may include, for example, visual and auditory anomalies exhibited by the participant that may affect the way a participant perceives stimuli. As just some examples, when a participant is rotated through space on the movement table and is presented with various stimuli such characteristics can include: a tendency by a participant to turn their head to one side possibly indicating that the vision abilities of the participant are not well established; the participant perceives that the light appears divided into two lights that may indicate a convergence problem; the participant is unable to direct a laser pointer at a stationary object may indicate a level of disorientation; the participant reports that they are not lying flat, but feels like they are “lying on a hill” may indicate a vestibular problem that affects posture and/or sense of posture; the participant exhibits a high degree of fear possibly indicating a weak vestibular, visual and auditory integration; the participant is restless and agitated, but becomes calm when an alphabet or number sequence is counted may indicate a degree of hyperplexia and where concrete sensations of movement have been overwhelming; the participant does not see the light moving may indicate that the vestibular system is not influencing the visual system normally possibly inhibiting the post-rotatory nystagmus reflex; the participant has difficulty following the light when rotated horizontally and crossing the midline can indicate a horizontal eye muscle problem; the participant has difficulty following the light when rotated vertically and crossing the eye level which may be an indication of a vertical eye muscle problem (e.g., Brown's syndrome, where there is a limitation of upward gaze); the participant is seen to develop an oral fixation, such as beginning to suck their thumb or to chew on something may indicate that the sensory program has “switched on” the oral/motor system; the participant becomes receptive to the auditory stimulation, this appears to make listening come alive, by stimulating the stirrup bone and the trapezius with gated reflexes to the muscles involved with speech; and the participant becomes itchy and scratchy around the head and rubs eyes, this is the start of breaking down the tactile defensiveness, and normalizes hypo-reactions to pain (this can be manifest in a change from not wanting to be touched to wanting to be touched). Various characteristics can be identified before the participant is exposed to any stimuli (pre-sensory), or after the participant is already exposed to some stimuli (post-sensory). One or more of these characteristics are provided to the remote server (block 320). At the remote server, the characteristics are used to identify a sensory program particularly suited to the characteristics.

As just one example, when the visual field shows anomaly of difficulty moving eye or eyes upward to denote visual stimulus, the movement table can be programmed to move the participant anterial-posterial (head to toe) across the horizon line when the extrinsic eye muscle is inhibited from being raised to gaze. As some other examples, a left ear threshold above the right ear threshold can be relied upon to remotely cause an increase (or weighting) of the right ear volume to establish correct right ear dominance, or where an inhibition in initiation and execution of speech in response to auditory presentations a binaural rather than monaural presentation of music may be indicated. As yet another example, when a visual field is constricted to less than five degrees, a thirty minute light session rather than a twenty minute light session may be selected by a trained professional located remote from the participant.

The appropriate sensory program is then received from the remote server (block 330). In some cases, the sensory program includes an audio input portion (block 333), a visual input portion (block 335), and a movement input portion (block 337). Such input portions can be tailored for commanding a respective one of table control system 270, audio control system 280 and light control system 290.

As one example, the light input portion can be tailored to select between a number of color programs that can be displayed via visual input device 110. In one embodiment, two hundred color programs are available providing different color sequences displayed across a twenty minute interval followed by ten minutes of darkness. In other embodiments, six hundred color programs are available providing different color sequences displayed across a twenty minute interval followed by ten minutes of darkness, and then followed by an additional twenty minute light interval and ten minute dark interval. In yet other embodiments, three hundred color programs are available that provide different color sequences during a thirty minute interval without an intervening dark interval. As another example, the movement input portion can command a movement table to operate at a particular rate during the time when the light program is being presented, and to stop movement when the light program completes.

The received sensory program is implemented (block 340). This includes, for example, applying the audio input via commands to audio control system 280 (block 343), applying visual input via commands to light control system 290 (block 345), and/or applying movement input via table control system 270 (block 347). After applying the stimuli, various characteristics about the participant are identified (post-sensory) (block 350). These characteristics can be the same as in other evaluations of the participant, or can include different characteristics that are observed. As just one example, it may be determined if the visual field of the participant has expanded indicating that more photocurrent is travelling along the optic nerve. These newly observed characteristics can then be communicated to the remote server (block 360).

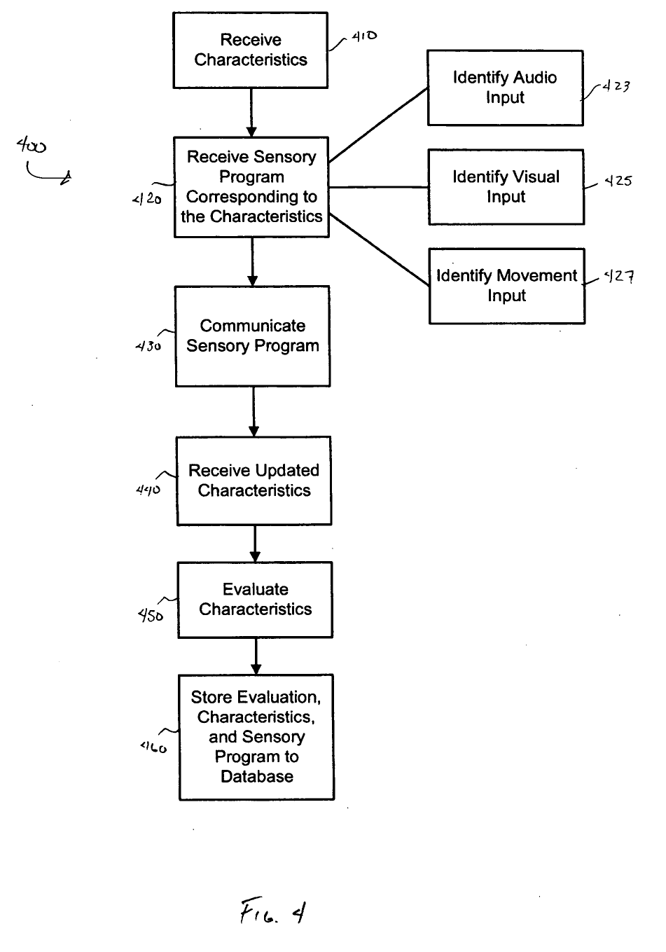

Turning to FIG. 4, a flow diagram 400 illustrating a method for delivering sensory stimuli in relation to an evaluation is depicted. Following flow diagram 400, one or more participant characteristics are received (block 410). These participant characteristics are used to select one of a plurality of sensory programs corresponding to the characteristics, and the selected sensory program is received (block 420). The selected sensory program can include one or more of an audio input portion (block 423), a visual input portion (block 425), and a movement input portion (block 427). Such input portions can be tailored for commanding a respective one of table control system 270, audio control system 280 and light control system 290.

The sensory program is communicated to a controller associated with a sensory input system (block 430). This can be done, for example, from a remote server via a communication network. The sensory program can then be executed by one or more computer processors associated with the sensory input system such that desired stimuli are presented to a participant. Characteristics of the participant are again obtained (post-sensory characteristics) and these characteristics are received (block 440). In some cases, the same characteristics are received in both blocks 410 and 440, while in other cases different characteristics are received. The received characteristics are evaluated (block 450). Such an evaluation can, for example, determine any changes in the participant that can be attributed to application of the sensory program. In some cases, this evaluation is performed by a technician or other trained personnel located remote from the sensory input system. In other cases, the evaluation could be performed automatically by comparing the received characteristics with a list of sensory programs corresponding to particular characteristics. The evaluation information, characteristics and sensory program selected are stored to a database (block 460).

As just one example, a twelve year old participant with a constricted visual field and uneven listening profile with a divergent peak (i.e., both ears moving in different sensitivity directions) at 8 KHz may be exposed on a movement table to a thirty minute light series and monaural acoustic program filtered at 12 KHz. Where the child becomes emotionally overwhelmed and is experiencing headaches, a switch to binaural modulation with a reduction in volume and change in light series may be indicated. With these changes, it may be found that the participant adapts more easily and successfully to the sensory program.

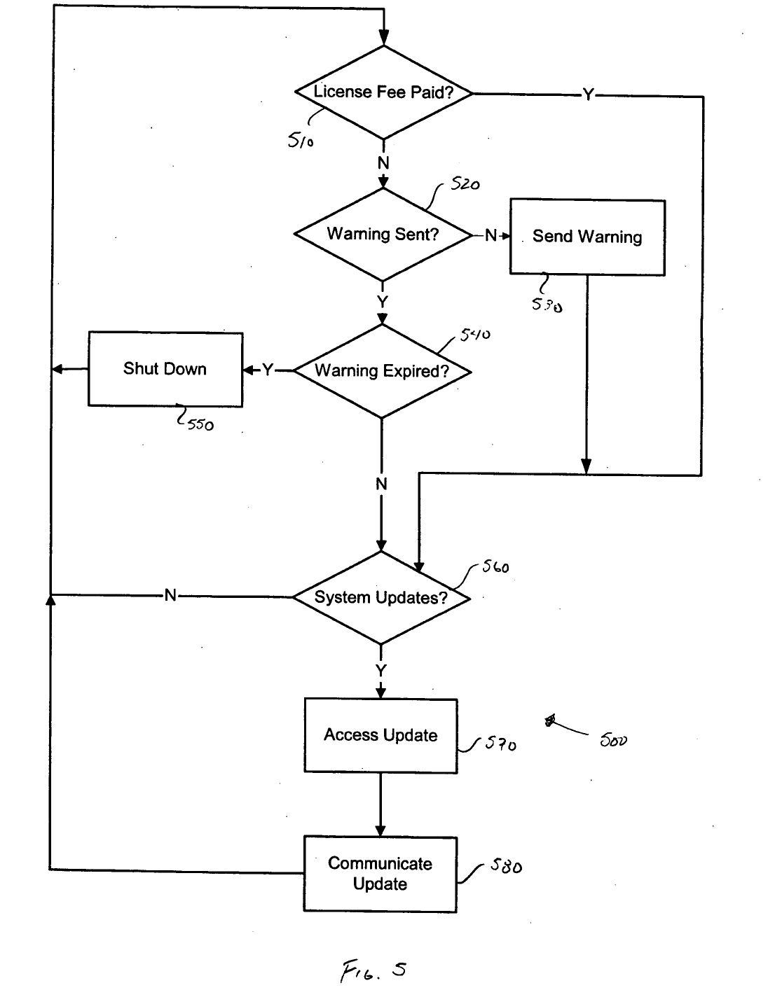

Turning to FIG. 5, a flow diagram 500 illustrates a method for remote use prevention in accordance with one or more embodiments of the present invention. Following flow diagram 500, it is determined if a license fee allowing operation of a sensory input system has been paid (block 510). Where the license fee has been paid (block 510), it is determined if any system updates are available (block 560). Where system updates are available (block 560), the appropriate updates are accessed (block 570) and communicated or downloaded to a controller associated with the remote sensory input system (block 580).

Alternatively, where the appropriate license fee has not been paid (block 510), it is determined if a user of the sensory input system has been warned of the failure to pay the fee (block 520). Where the user has not yet been warned (block 520), a warning is sent (block 530) and system updates are performed as previously described in relation to block 560-580. On the other hand, where a warning has been issued (block 520), it is determined if the warning has expired (block 540). If the warning has expired, a command is issued causing precluding operation of one or more elements of the sensory input system (block 550).

The invention has now been described in detail for purposes of clarity and understanding. However, it will be appreciated that certain changes and modifications may be practiced within the scope of the appended claims. Thus, although the invention is described with reference to specific embodiments and figures thereof, the embodiments and figures are merely illustrative, and not limiting of the invention. Rather, the scope of the invention is to be determined solely by the appended claims.

What is claimed is:

1. A system for providing multi-sensory stimuli to a participant, the system comprising:
a trochoidal movement table;
a light emitting visual display;
an audio device;
a controller including a processor and a computer readable medium;
wherein the controller is electrically coupled to the trochoidal movement table and the light emitting display;
wherein the controller is operable to receive a sensory program from a remote server and to store the sensory program to the computer readable medium; and
wherein the sensory program includes instructions executable by the processor to:
cause the trochoidal movement table to move in one of at least two selectable directions; and
cause the light emitting visual display to emit a visual stimulus.

2. The system of claim 1, wherein the controller is further operable to receive an operational key from the remote server, and wherein the operational key is operable to permit operation of at least one of the trochoidal movement table, the light emitting visual display, and the audio device.

3. The system of claim 1, wherein the sensory program is a first sensory program, and wherein the controller is operable to receive a second sensory program in response to a characteristic of the participant.

4. The system of claim 3, wherein the characteristic of the participant is evaluated upon interaction of the participant to stimuli produced in relation to the first sensory program.

5. A system for providing multi-sensory stimuli to a participant, the system comprising:
a movement table wherein the movement table is operable to support the participant;
a light emitting visual display, wherein the light emitting display is disposed in relation to the movement table such that the participant can view the light emitting display when positioned on the movement table;
an audio device, wherein the audio device is disposed in relation to the movement table such that a participant is exposed to an audio stimulus from the audio device when positioned on the movement table;
a controller including a processor and a computer readable medium;
wherein the controller is communicably coupled to the movement table, the light emitting display, and the audio device; and
wherein the controller is operable to receive an operational key from a remote server, and wherein the operational key is operable to permit operation of at least one of the movement table, the light emitting visual display, and the audio device.

6. The system of claim 5, wherein the controller is further operable to receive a sensory program from the remote server and to store the sensory program to the computer readable medium; and wherein the sensory program includes instructions executable by the processor to:
cause the movement table to move at a defined rate;
cause the audio device to emit an audible stimulus; and
cause the light emitting visual display to emit a visual stimulus.

7. A method for delivering a multi-sensory experience, the method comprising:
providing a movement table;
providing a light emitting visual display;
providing an audio device;
providing a controller, wherein the controller is electrically connected to at least one of the movement table, the audio device, and the light emitting visual display; and
delivering a command to the controller via a communication network, wherein the command at least in part controls the stimuli delivered by at least one of the movement table, the light emitting display, and the audio device.

8. The method of claim 7, wherein the command is operable to preclude use of at least one of the movement table, the light emitting visual display and the audio device.

9. The method of claim 7, wherein the command is operable to cause a modulated musical sound to emanate from the audio device.

10. The method of claim 7, wherein the command is operable to cause a series of uniform colors to display via the light emitting display.

11. The method of claim 7, wherein the command is operable to control a rate at which the movement table moves.

12. A multi-sensory introduction system, wherein the system comprises:
a movement table, wherein the movement table is operable to introduce a movement sense to a participant disposed on the table;
an auditory input device, wherein the auditory input device is operable to introduce an audio sense to the participant;
a visual input device, wherein the visual input device is operable to introduce a visual sense to the participant; and
a controller communicably coupled to at least one of the movement table, the auditory device, and the visual input device, and wherein the controller is operable to receive a command associated with the at least one of the movement table, the auditory device, and the visual input device from a remote server.

13. The multi-sensory introduction system of claim 12, wherein the movement table is a trochoidal movement table.

14. The multi-sensory introduction system of claim 13, wherein the trochoidal movement table is controlled by the controller, wherein the controller receives a movement command from the remote server, and wherein the movement sense is in accordance with the movement command.

15. The multi-sensory introduction system of claim 13, wherein the trochoidal table maintains the head of the participant fixed in relation to the body of the participant.

16. The multi-sensory introduction system of claim 12, wherein the auditory input device is further operable to receive a song and to segregate the song into at least one song segment, and wherein the audio sense introduced to the participant includes the at least one song segment.

17. The multi-sensory introduction system of claim 12, wherein the auditory input device is further operable to receive a song and to modify the song into modulated musical sound, and wherein the modulated musical sound is the audio sense introduced to the participant.

18. The multi-sensory introduction system of claim 12, wherein the controller is operable to receive a song from the remote server, and wherein the audio sense includes at least a portion of the song.

19. The multi-sensory introduction system of claim 12, wherein the visual input device is further operable to receive a light sequence command, and wherein the visual sense includes one or more light outputs in accordance with the light sequence command.

20. The multi-sensory introduction system of claim 19, wherein the light sequence command is received from the remote server.

21. The multi-sensory introduction system of claim 12, wherein the controller is further operable to prevent operation of at least one of all of the movement table, the auditory device, and the visual input device in accordance with an operational command.

22. The multi-sensory introduction system of claim 21 wherein the operational command is received from the remote server.

23. The multi-sensory introduction system of claim 12, wherein the controller is further operable to provide a status update to the remote server, and wherein the status update is selected from a group consisting of: a system hardware performance, a system hardware status, and a hardware error.

24. A server based sensory introduction system, wherein the system comprises:
a server communicably coupled to a remote multi-sensory introduction system, wherein the serer includes a processor and a computer readable medium, and wherein the computer readable medium includes instructions executable by the processor to:
select a sensory program, wherein the sensory program includes at least two sensory commands selected from a group of sensory commands consisting of: an auditory command, a movement command, and a visual command; and
communicate the sensory program to the remote multi-sensory introduction system.

25. The server based sensory introduction system of claim 24, wherein the computer readable medium further includes instructions executable by the processor to:
check a compliance characteristic of the remote multi-sensory introduction system; and
communicate an operational command to the remote multi-sensory introduction system based at least in part on the compliance characteristic.

26. The server based sensory introduction system of claim 25, wherein the remote multi-sensory introduction system includes a controller, and wherein the operational command is implemented by the controller to restrict operation of the remote multi-sensory introduction system.

27. The server based sensory introduction system of claim 25, wherein the sensory program is a first sensory program, wherein the remote multi-sensory introduction system is a first remote multi-sensory introduction system, wherein the server based sensory introduction system includes a second remote multi-sensory introduction system, and wherein the computer readable medium further includes instructions executable by the processor to:
select a second sensory program, wherein the second sensory program includes at least two sensory commands selected from a group of sensory commands consisting of: an auditory command, a movement command, and a visual command; and
communicate the second sensory program to the remote multi-sensory introduction system.

28. The server based sensory introduction system of claim 24, wherein the remote multi-sensory introduction system includes:
a remote processor;
a movement table;
an auditory input device;
a visual input device; and
wherein the sensory program is executable by the remote processor to:
cause rhythmic movement of the movement table;
cause modulated musical sound output from the auditory input device;
cause a colored light output from the visual input device; and
wherein the rhythmic movement, the modulated musical sound output and the colored light output are delivered in concert.

29. The server based sensory introduction system of claim 24, wherein the computer readable medium further includes instructions executable by the processor to:
receive a participant characteristic; and
wherein selecting the sensory program is based at least in part on the participant characteristic.

30. The server based sensory introduction system of claim 29, wherein the participant characteristic is a pre-sensory characteristic, and wherein the computer readable medium further includes instructions executable by the processor to:
receive a post-sensory characteristic; and
evaluating the sensory program based at least in part on the post-sensory characteristic.

31. The server based sensory introduction system of claim 30, wherein the sensory program is a first sensory program, and wherein the computer readable medium further includes instructions executable by the processor to:
select a second sensory program based at least in part on the post-sensory characteristic; and
communicate the second sensory program to the remote multi-sensory introduction system.
